(19) United States
(12) Patent Application Publication
(10) Pub. No.: US 2025/0354223 A1
(43) Pub. Date: Nov. 20, 2025

(54) METHODS AND KITS FOR DETERMINING ANTIBIOTIC SUSCEPTIBILITY

(71) Applicant: Siemens Healthcare Diagnostics Inc., Tarrytown, NY (US)

(72) Inventors: Nivedita MITRA, Bangalore (IN); Ramya V M, Bangalore (IN)

(21) Appl. No.: 18/867,522

(22) PCT Filed: May 31, 2023
(86) PCT No.: PCT/US2023/067667

§ 371 Date: Nov. 20, 2024

(30) Foreign Application Priority Data
May 31, 2022 (IN) 202241031205

(51) Int. Cl. C12Q 1/689

(52) U.S. Cl. CPC C12Q 1/689; C12Q 2600/156; C12Q 2600/158

(57) ABSTRACT

The present disclosure relates to a method of determining antibiotic susceptibility of a micro-organism in a sample, the method including: receiving the sample containing the micro-organisms; incubating at least one first portion of the sample with at least one antibiotic and at least one second portion of the sample with no antibiotic; extracting nucleic acid from the at least one first portion of the sample and the at least one second portion of the sample, wherein the nucleic acid is associated with the micro-organisms present in the sample; amplifying the extracted nucleic acid from the at least one first portion and the at least one second portion of the sample; and obtaining the antibiotic susceptibility information associated with the micro-organism from the amplified nucleic acid from the at least one first portion and the at least one second portion of the sample.

### CROSS-REFERENCE TO RELATED APPLICATIONS

This application claims priority or the benefit of I.N. patent application no. 202241031205 filed on 31 May 2022, the contents of which are fully incorporated herein by reference.

### FIELD OF THE INVENTION

The present disclosure relates to the field of analysis of a sample and more particularly to the field of determining antibiotic susceptibility of micro-organisms.

### BACKGROUND OF THE INVENTION

There is a steady rise in the rate of antibiotic resistance in micro-organisms. This makes it necessary to administer an appropriate and adequate antibiotic therapy to a patient. Current methods of determining antimicrobial resistance are slow and do not give complete information for antibiotic susceptibility.

Traditionally, microbial culture-based methods are used for determination of antibiotic susceptibility or resistance, and it takes about 3-4 days for a clinically actionable result. Polymerase chain reaction (PCR)-based methods may also be used in determining antimicrobial resistance information. However, the turnaround time for such genotypic methods is about two-three hours and the amount of antimicrobial resistance information obtained is limited. These tests need continuous development and improvement to include the evolving genetic signatures from resistant micro-organisms.

Prior-art-of-interest includes U.S. Patent Publication No. 20170058314 to Zhang et al., U.S. Pat. No. 9,677,109, and U.S. Patent Publication No. 20230054472, entitled Compositions, devices and methods for diagnosing and treating infectious disease to Varma et al. (all of which are entirely incorporated herein by reference).

Therefore, there is a continuing need for effective ways of determining antibiotic susceptibility of micro-organisms which is fast and provide complete information associated with antimicrobial resistance to aid physician in taking timely clinical decisions.

### SUMMARY OF THE INVENTION

Methods, kits, apparatuses for determining antibiotic susceptibility of one or more micro-organisms are provided herein. In embodiments, a method for determining antibiotic susceptibility of one or more bacterial micro-organism in a sample, such as a biological sample is disclosed.

In embodiments, a method of the present disclosure includes receiving a sample containing one or more micro-organisms. Additionally, the method includes incubating at least one first portion of the sample with at least one antibiotic and at least one second portion of the sample with no antibiotic. The method further includes extracting nucleic acid from the at least one first portion of the sample and the at least one second portion of the sample, wherein the nucleic acid is associated with the micro-organisms present in the sample. In embodiments, the extracted nucleic acid is amplified, and the antibiotic susceptibility information associated with the micro-organism is obtained from the differences observed in the results from amplified nucleic acid from with and without antibiotic reactions.

In embodiments, the present disclosure includes a method of determining antibiotic susceptibility of a micro-organism in a sample, the method including: receiving the sample containing the micro-organisms; incubating at least one first portion of the sample with at least one antibiotic and at least one second portion of the sample with no antibiotic; contacting the at least one first portion and/or at least one second portion with one or more intercalating dyes; extracting nucleic acid from the at least one first portion of the sample and the at least one second portion of the sample, wherein the nucleic acid is associated with the micro-organisms present in the sample; amplifying the extracted nucleic acid from the at least one first portion and the at least one second portion of the sample; and obtaining the antibiotic susceptibility information associated with the micro-organism from the amplified nucleic acid from the at least one first portion and the at least one second portion of the sample. In embodiments, the intercalating dye may be ethidium monoazide, ethidium monoazide bromide, propidium monoazide, isomers thereof, and the like.

In embodiments, a method of the present disclosure includes incubating or culturing at least one first portion of a biological sample containing one or more bacterial microbes with at least one antibiotic and at least one second portion of the sample with no antibiotic; extracting nucleic acid from the at least one first portion of the sample and the at least one second portion of the sample, wherein the nucleic acid is associated with the micro-organisms present in the sample; and amplifying the extracted nucleic acid such that the antibiotic susceptibility information associated with the micro-organism is obtained from the differences observed in the results from amplified nucleic acid from with and without antibiotic reactions.

In embodiments, a method of the present disclosure includes incubating or culturing at least one first portion of a biological sample containing one or more bacterial microbes with at least one antibiotic and at least one second portion of the sample with no antibiotic; extracting nucleic acid from the at least one first portion of the sample and the at least one second portion of the sample, wherein the nucleic acid is associated with the micro-organisms present in the sample; contacting the extracted nucleic acid with one or more intercalating dyes; and amplifying the extracted nucleic acid such that the antibiotic susceptibility information associated with the micro-organism is obtained from the differences observed in the results from amplified nucleic acid from with and without antibiotic reactions. In embodiments, contacting the extracted nucleic acid with one or more intercalating dyes further includes applying light (such as UV light) under conditions sufficient for the extracted nucleic acid and the intercalating dye to be associated with one another, or form a nucleic acid/intercalating dye complex. For example, the intercalating dye may be ethidium monoazide, ethidium monoazide bromide, propidium monoazide, isomers thereof, and the like.

In embodiments, the present disclosure includes a kit for determining antibiotic susceptibility of a micro-organism in a sample. In embodiments, a kit includes one or more antibiotics. The antibiotics may be provided in defined amounts such that effective results are achieved. The kit may further include reactions volumes specific to growth conditions of the micro-organisms that may be present in the sample. Additionally, the kit may include media for growth of micro-organisms present in the sample. The volume of growth media may be defined to achieve optimum results of micro-organism growth. Further, in embodiments, the kit includes nucleic acid extraction-based reagents and nucleic acid amplification-based reagents. In particular, the nucleic acid amplification reagents may include one or more primers targeting 16srRNA and/or 16srDNA of the micro-organism in the sample. Additionally, the nucleic acid amplification kit may also include one or more TaqMan probes. In a further embodiment, the kit includes a polymerase chain reaction-based melt analysis software. In yet another embodiment, the kit includes at least one intercalating dye. For example, the dye may be ethidium monoazide, ethidium monoazide bromide, propidium monoazide, isomers thereof, and the like.

In embodiments, the present disclosure includes a kit for determining antibiotic susceptibility of a micro-organism in a sample, the kit including: one or more antibiotics; growth media for micro-organisms; reagents associated with nucleic acid extraction; reagents associated with nucleic acid amplification; and polymerase chain reaction-based melt analysis software. In embodiments, the kit includes instructions for using the kit and/or software. In embodiments, the kit includes one or more intercalating dyes.

In embodiments, the present disclosure includes an article of manufacture, such as a system or component thereof including a non-transitory computer-readable medium with instructions encoded thereon, the instructions configured to cause one or more processors to perform a method of determining antibiotic susceptibility of a micro-organism in a sample, the method including: incubating at least one first portion of a sample containing micro-organisms with at least one antibiotic and at least one second portion of the sample with no antibiotic; extracting nucleic acid from the at least one first portion of the sample and the at least one second portion of the sample, wherein the nucleic acid is associated with the micro-organisms present in the sample; amplifying the extracted nucleic acid from the at least one first portion and the at least one second portion of the sample; and obtaining the antibiotic susceptibility information associated with the micro-organism from the amplified nucleic acid from the at least one first portion and the at least one second portion of the sample. In embodiments, the method further includes contacting the sample, portions of the sample, or extracted nucleic acids with one or more intercalating dyes, prior to amplifying.

In embodiments the present disclosure includes a non-transient computer readable medium with instructions encoded thereon, the instructions configured to cause one or more processors to perform a method of determining antibiotic susceptibility of a micro-organism in a sample, the method including: incubating at least one first portion of a sample containing micro-organisms with at least one antibiotic and at least one second portion of the sample with no antibiotic; extracting nucleic acid from the at least one first portion of the sample and the at least one second portion of the sample, wherein the nucleic acid is associated with the micro-organisms present in the sample; contacting the nucleic acid with one or more intercalating dyes under conditions to form a nucleic acid/intercalating dye complex; amplifying the extracted nucleic acid from the at least one first portion and the at least one second portion of the sample; and obtaining the antibiotic susceptibility information associated with the micro-organism from the amplified nucleic acid from the at least one first portion and the at least one second portion of the sample.

This summary is provided to introduce a selection of concepts in a simplified form that are further described below in the following description. It is not intended to identify features or essential features of the claimed subject matter. Furthermore, the claimed subject matter is not limited to implementations that solve any or all disadvantages noted in any part of this disclosure.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 illustrates a method of determining antibiotic susceptibility of micro-organisms, according to an embodiment.

FIG. 2 illustrates a method of determining antibiotic susceptibility of micro-organisms, according to another embodiment.

FIG. 3 illustrates a method of determining antibiotic susceptibility of micro-organisms, according to yet another embodiment.

FIGS. 4A and 4B illustrate a graphical representations of cycle threshold values associated with samples exposed to antibiotic only and a combination on antibiotics and an intercalating dye.

FIG. 5 depicts a system for determining bacterial susceptibility, according to an embodiment.

FIG. 6 depicts a method of determining antibiotic susceptibility of micro-organisms, according to yet another embodiment including EMA.

FIG. 7 depicts a method of determining antibiotic susceptibility of micro-organisms, according to yet another embodiment.

FIG. 8 depicts a method of determining antibiotic susceptibility of micro-organisms, according to yet another embodiment.

FIG. 9 depicts a method of determining antibiotic susceptibility of micro-organisms, according to yet another embodiment.

FIG. 10 depicts data related to example 1.

FIG. 11 depicts data relating to E. coli and ciprofloxacin obtained according to an embodiment of the present disclosure.

FIG. 12 depicts data relating to E. coli obtained according to an embodiment of the present disclosure.

FIG. 13 depicts data relating to pseudomonas with ciprofloxacin and chloramphenicol obtained according to an embodiment of the present disclosure.

FIG. 14 depicts data related to example 2.

FIG. 15 depicts antibiotic incubation times in accordance with embodiments of the present disclosure.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, devices, kits, and/or methods disclosed herein can be made and executed without undue experimentation in light of the present disclosure. While the compositions, devices, kits, and/or methods have been described in terms of particular embodiments, it will be apparent to those of skill in the art that variations may be applied to the compositions, devices, kits, and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

Embodiments of the present disclosure beneficially provide robust rapid drug susceptibility testing and drug screening. Various features and advantages that may be achieved by the present disclosure will be appreciated from the discussion herein and may include one or more of the following: (1) suitability for use with prokaryotic cells which may poorly grow in culture; and (2) use of targeted drug concentrations for inhibiting organism growth. In embodiments, susceptibility includes instances in which an antimicrobial agent has an inhibitory effect on the growth of a microorganism or a lethal effect on the microorganism. In embodiments, susceptibility includes instances in which an antimicrobial agent has a lethal effect on the microorganism. Susceptibility further includes the concept of a minimum inhibitory concentration (“MIC”) of an antimicrobial agent, as a concentration of an antimicrobial agent that will arrest growth of a microorganism. Identification of susceptibility, or a lack of susceptibility for example, using the system and method described herein, may provide information that may be useful to a clinician or health care decision maker in making a decision regarding antimicrobial agent therapy for a patient in need thereof. In embodiments, reduced growth or functional response in the presence of the antibiotic agent relative to a control indicates that the microbe is susceptible to the antibiotic agent tested.

### DEFINITIONS

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural references unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and, unless explicitly stated otherwise, is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

An “analyte” is a nucleic acid macromolecule that is capable of being recognized by an analyte-specific binding partner. In embodiments, an analyte refers to a nucleic acid macromolecule that is capable of being recognized by an analyte-specific binding partner such as (but not limited to) a DNA or RNA segment, strand or oligomer or portion thereof that is complimentary or substantially complimentary to the analyte such that it is able to bind thereto.

As used herein, the term “antibiotic” refers to a compound that has the ability to kill or inhibit the growth of bacteria. In some embodiments the term “antibiotic” refers to a compound that has the ability to kill or inhibit the growth of bacteria particularly bacteria selected from a genus including, but not limited to, Borrelia, Staphylococcus, Escherichia, Klebsiella, Acinetobacter, and Mycobacterium. In embodiments, an antimicrobial is an agent that kills microorganisms or inhibits their growth. In embodiments, antimicrobials such as medicines can be grouped according to the microorganisms they act primarily against. For example, antibiotics are used against bacteria. As used herein, the term ‘beta-lactam” or “beta-lactam antibiotic” refers to an antibiotic with a beta-lactam ring as part of its core structure, such as penicillin and penicillin derivatives (penams), cephalosporins (cephems), monobactams, and carbapenems. While these antibiotics work by inhibiting bacterial cell wall biosynthesis, other antibiotics such as protein synthesis inhibitors (tetracyclines, aminoglycosides, macrolides, etc), nucleic acid synthesis inhibitors (fluoroquinolones, rifamycins etc) and antimetabolite sulfa drugs can also be used similarly for antibiotic susceptibility testing with the methodology described herein.

As used herein, the term “bacterial infection” means the invasion by, multiplication and/or presence of a bacteria in a cell or a subject.

As used herein, the term “cell culture” or “culture” includes a reference to a population of cells in culture. In some embodiments a cell culture or culture may refer to the cells being in a medium conducive for growth of the cells and to optionally further the cells growing in the medium. Thus, in some embodiments the term “bacterial culture” or “culture” may refer to bacteria growing or incubating in a medium conducive for growth of those bacteria. The bacterial culture can be found in any type of container, such as a flask, a tube, a microwell plate, or array, and the like. Generally, bacteria have different phases of growth. When a population of bacteria first enters a high-nutrient environment that allows growth, the cells need to adapt to their new environment. The first phase of growth is the lag phase, a period of slow growth when the cells are adapting to the high-nutrient environment and preparing for fast growth. The lag phase has high biosynthesis rates, as proteins necessary for rapid growth are produced. The second phase of growth is the log phase, also known as the logarithmic or exponential phase, in which the bacteria undergo rapid exponential growth. During log phase, nutrients are metabolized at maximum speed until one of the nutrients is depleted and starts limiting growth. The third phase of growth is the stationary phase and is caused by depleted nutrients. In some embodiments, a bacterial culture in “stationary phase” means that the bacteria in the culture have an approximately equal growth rate and death rate. As used herein, the term “growing forms” of bacteria generally refers to bacteria that are in lag or log phase and not in stationary phase. In some embodiments, the stationary phase bacterial culture has been grown for approximately 7 days. In other embodiments, the stationary phase bacterial culture includes non-replicating persister cells. By “non-replicating persister cells,” it is meant bacterial cells that enter a state in which they stop replicating and are able to tolerate antibiotics.

The term “contacting” as used herein refers to any action that results in at least one compound or component of the presently disclosed subject matter physically contacting at least one cell (e.g. bacterial cell) or the environment in which at least one cell (e.g. bacterial cell) resides (e.g., a culture medium or sample).

As used herein, the terms “disease” and “disorder” are used interchangeably to refer to a condition in a subject including a harmful deviation from the normal structural or functional state of an organism. Non-limiting examples of diseases/disorders include a subject having one or more bacterial infections, or sepsis

Detection agent: The term “detection agent” as used herein refers to any element, molecule, functional group, compound, fragment or moiety that is detectable. In some embodiments, a detection agent is provided or utilized alone. In some embodiments, a detection agent is provided and/or utilized in association with (e.g., joined to) another agent. Examples of detection agents include, but are not limited to: various ligands, fluorescent dyes, chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes, colorimetric labels (such as, for example, dyes, colloidal gold, and the like), biotin, dioxigenin, haptens, and proteins for which antisera or monoclonal antibodies are available.

Diagnostic test: As used herein, “diagnostic test” is a step or series of steps that is or has been performed to attain information that is useful in determining whether a patient has a disease, disorder or condition and/or in classifying a disease, disorder or condition into a phenotypic category or any category having significance with regard to prognosis of a disease, disorder or condition, or likely response to treatment (either treatment in general or any particular treatment) of a disease, disorder or condition. Similarly, “diagnosis” refers to providing any type of diagnostic information, including, but not limited to, whether a subject is likely to have or develop a disease, disorder or condition, state, staging or characteristic of a disease, disorder or condition as manifested in the subject, information related to the nature or classification of a tumor, information related to prognosis and/or information useful in selecting an appropriate treatment or additional diagnostic testing. Selection of treatment may include the choice of a particular therapeutic agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc. Selection of additional diagnostic testing may include more specific testing for a given disease, disorder, or condition.

Hybridization: The term “hybridization” refers to the physical property of single-stranded nucleic acid molecules (e.g., DNA or RNA) to anneal to complementary nucleic acid molecules Hybridization can typically be assessed in a variety of contexts—including where interacting nucleic acid molecules are studied in isolation or in the context of more complex systems (e.g., while covalently or otherwise associated with a carrier entity and/or in a biological system or cell). Hybridization techniques, and methods for evaluating hybridization, are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

The terms “labeled” and “labeled with a detectable agent (or moiety)” are used herein interchangeably to specify that an entity (e.g., a target sequence) can be visualized, e.g., directly or following hybridization to another entity that includes a detectable agent or moiety. In embodiments, the detectable agent or moiety is selected such that it generates a signal which can be measured and whose intensity is related to (e.g., proportional to) the amount of the entity of interest (e.g., a target sequence). Methods for labeling nucleic acid molecules are well-known in the art. In some embodiments, labeled nucleic acids can be prepared by incorporation of, or conjugation to, a label that is directly or indirectly detectable by spectroscopic, photochemical, biochemical, immunochemical, electrical, optical, or chemical means.

In some embodiments, the term “oligonucleotide” is used herein to denote a polynucleotide that includes between about 5 and about 150 nucleotides, e.g., between about 10 and about 100 nucleotides, between about 15 and about 75 nucleotides, or between about 15 and about 50 nucleotides. Throughout the specification, whenever an oligonucleotide is represented by a sequence of letters (chosen, for example, from the four base letters: A, C, G, and T, which denote adenosine, cytidine, guanosine, and thymidine, respectively), the nucleotides are presented in the 5′ to 3′ order from the left to the right. A “polynucleotide sequence” refers to the sequence of nucleotide monomers along the polymer. Unless denoted otherwise, whenever a polynucleotide sequence is represented it will be understood that the nucleotides are in 5′ to 3′ orientation from left to right.

The term “nucleic acid” as used herein means a nucleobase polymer having a backbone of alternating sugar and phosphate units in DNA and RNA. In embodiments, “Nucleic acid” and “polynucleotide” are considered to be equivalent and interchangeable. Nucleic acids are commonly in the form of DNA or RNA. In some embodiments, the terms “nucleic acid”, “nucleic acid molecule”, “polynucleotide” or “oligonucleotide” are used herein interchangeably. They refer to polymers of nucleotide monomers or analogs thereof, such as deoxyribonucleic acid (DNA) and ribonucleic acid (RNA). The nucleotides may be genomic, synthetic or semi-synthetic in origin. Unless otherwise stated, the terms encompass nucleic acid-like structures with synthetic backbones, as well as amplification products. As will be appreciated by one skilled in the art, the length of these polymers (i.e., the number of nucleotides it contains) can vary widely, often depending on their intended function or use. Polynucleotides can be linear, branched linear, or circular molecules. In embodiments, polynucleotides also have associated counter ions, such as H⁺, NH₄⁺, trialkylammonium, Mg₂⁺, Na⁺ and the like. A polynucleotide may be composed entirely of deoxyribonucleotides, entirely of ribonucleotides, or chimeric mixtures thereof. Polynucleotides may be composed of internucleotide nucleobase and sugar analogs.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrase “associated with” includes both direct association of two moieties to one another as well as indirect association of two moieties to one another. Non-limiting examples of associations include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety.

The term “biological fluid sample” as used herein will be understood to include any liquid test sample that may be obtained from a patient and utilized in accordance with the present disclosure. Examples of biological fluid samples that may be utilized include, but are not limited to, whole blood or any portion thereof (i.e., plasma or serum), saliva, sputum, mucus, nasal, nasopharyngeal, anterior nasal, oropharyngeal, tracheal, bronchoalveolar, cerebrospinal fluid (CSF), intestinal fluid, intraperitoneal fluid, cystic fluid, sweat, interstitial fluid, tears, combinations thereof, and the like.

As used herein, the term “volume” as it relates to the liquid test samples utilized in accordance with the present disclosure typically refers to a volume of liquid test sample in a range of from about 0.1 μl to about 100 μl, or a range of from about 1 μl to about 75 μl, or a range of from about 2 μl to about 60 μl, or a value less than or equal to about 50 μl, or the like.

The term “patient” as utilized herein includes human and veterinary subjects. In certain non-limiting embodiments, a patient is a mammal. In certain other non-limiting embodiments, the patient is a human. The term “mammal” for purposes of diagnosis/treatment refers to any animal classified as a mammal, including human, domestic and farm animals, nonhuman primates, and zoo, sports, or pet animals, such as dogs, horses, cats, cows, etc.

A “health care provider” or “health care decision maker” includes any individual authorized to diagnose or treat a patient, or to assist in the diagnosis or treatment of a patient. In the context of identifying useful new drugs to treat disease, a health care provider can be an individual who is not authorized to diagnose or treat a patient, or to assist in the diagnosis or treatment of a patient.

The term “isolated”, as used herein, means a target, sample, polynucleotide, complex, nucleic acid or oligonucleotide, which by virtue of its origin or manipulation, is separated from at least some of the components with which it is naturally associated or with which it is associated when initially obtained.

“Point of care testing” refers to real time diagnostic testing that can be done in a rapid time frame so that the resulting test is performed faster than comparable tests that do not employ this system. Point of care testing can be performed rapidly and on site, such as in a doctor's office, at a bedside, in a stat laboratory, emergency room, or other such locales, particularly where rapid and accurate results are required. The patient can be present, but such presence is not required. Point of care includes, but is not limited to: emergency rooms, operating rooms, hospital laboratories and other clinical laboratories, doctor's offices, in the field, or in any situation in which a rapid and accurate result is desired.

The term “specific binding partner,” as used in particular (but not by way of limitation) herein in the term “target analyte-specific binding partner,” will be understood to refer to any molecule capable of specifically associating with the target analyte. For example, but not by way of limitation, the binding partner may be an antibody, a receptor, a ligand, aptamers, molecular imprinted polymers (i.e., inorganic matrices), combinations or derivatives thereof, as well as any other molecules capable of specific binding to the target analyte.

As used herein, the terms “inhibit”, “inhibits”, or “significant decrease” means to decrease, suppress, attenuate, diminish, or arrest, for example the growth and/or survival of cells (e.g. bacteria) in a culture or in a subject, by at least 10%, 20%, 30%, 40%, 50%, 60%, 70%, 80%, 90%, 95%, 98%, 99%, or even 100% compared to an untreated control culture or subject. Inhibiting “survival” of cells (e.g. bacteria) in this context refers to killing cells or reducing live cell count. In some embodiments, the growth of the cells is inhibited by more than approximately 50%. In other embodiments, the percentage of live cells in the culture after the treatment with the test compound is less than approximately 50% compared to the percentage of live cells in the control under identical conditions, but in the absence of the test compound. In still other embodiments, the stationary phase culture comprises non-growing cells such as non-replicating persister cells. Further, as used herein, the term “significant increase” means an increase by at least 10%, 20%, 30%, 40%, 50%, 60%, 70%, 80%, 90%. 95%, 98%, 99%, or even 100%.

Sample: As used herein, the term “sample” refers to a biological sample obtained or derived from a human subject, as described herein. In some embodiments, a biological sample includes biological tissue or fluid. In some embodiments, a biological sample may include blood; blood cells; tissue or fine needle biopsy samples; cell-containing body fluids; free floating nucleic acids; cerebrospinal fluid; lymph; tissue biopsy specimens; surgical specimens; other body fluids, secretions, and/or excretions; and/or cells therefrom In some embodiments, a biological sample includes cells obtained from an individual, e.g., from a human or animal subject. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood). In some embodiments, a sample is cardiac tissue obtained from the subject. In some embodiments, as will be clear from context, the term “sample” refers to a preparation that is obtained by processing (e.g., by removing one or more components of and/or by adding one or more agents to) a primary sample. For example, filtering using a semi-permeable membrane. As another example of sample processing, the sample may be a plasma sample that is treated with an anticoagulant selected from the group consisting of EDTA, heparin, and citrate. As another example of sample processing, the sample may be processed to isolate one or more proteins (e.g., by capturing proteins with one or more antibodies). A “processed sample” may include, for example, nucleic acids or polypeptides extracted from a sample or obtained by subjecting a primary sample to techniques such as amplification or reverse transcription of mRNA, isolation and/or purification of certain components.

Subject: As used herein, the term “subject” refers to an organism, for example, a mammal (e.g., a human). In some embodiments a human subject is an adult, adolescent, or pediatric subject. In some embodiments, a subject is at least 50, at least 55, at least 60, at least 65, at least 70, at least 75, or at least 80 years of age. In some embodiments, a subject is suffering from a disease, disorder or condition, e.g., a disease, disorder or condition that can be treated as provided herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient In some embodiments, a subject is an individual to whom diagnosis and/or therapy is and/or has been administered.

Threshold value: As used herein, the term “threshold value” refers to a value (or values) that are used as a reference to attain information on and/or classify the results of a measurement, for example, the results of a measurement attained in an assay. A threshold value can be determined based on one or more control samples. A threshold value can be determined prior to, concurrently with, or after the measurement of interest is taken. In some embodiments, a threshold value can be a range of values. In some embodiments, a threshold value can be a value (or range of values) reported in the relevant field (e.g., a value found in a standard table).

As used herein, the term “therapeutically effective amount” means the amount of compound that, when administered to a subject for treating or preventing a particular disorder, disease or condition, is sufficient to affect such treatment or prevention of that disorder, disease or condition. Dosages and therapeutically effective amounts may vary for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g., bioavailability, stability, potency, toxicity, etc.), and the particular disorder(s) the subject is suffering from. In addition, the therapeutically effective amount that is administered intravenously may depend on the subject's blood parameters e.g., lipid profile, insulin levels, glycemia or liver metabolism. The therapeutically effective amount will also vary according to the severity of the disease state, organ function, or underlying disease or complications. Such appropriate doses may be determined using any available assays. When one or more of the compounds or therapeutic agents is to be administered to humans, a physician may for example, prescribe a relatively low dose at first, subsequently increasing the dose until an appropriate response is obtained.

The terms “treatment” or “treating” of a subject includes the application or administration of a compound to a subject with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being. In embodiments, the term “treating” means reducing or ameliorating the progression, severity, and/or duration of infection. In embodiments, the term “treating” means reducing or ameliorating the progression, severity, and/or duration of a bacterial, infection or ameliorating one or more symptoms of a bacterial infection caused by administration of one or more therapies (e.g., one or more therapeutic agents). In a particular embodiment, the term “treatment” means to ameliorate measurable physical parameters of a bacterial infection. In embodiments, the term “treating” means reducing or ameliorating the progression, severity, and/or duration of sepsis, or ameliorating one or more symptoms of a sepsis caused by administration of one or more therapies (e.g., one or more therapeutic agents). In a particular embodiment, the term “treatment” means to ameliorate measurable physical parameters of sepsis. In embodiments, “treating” changes the natural or presenting state of a subject.

Hereinafter, embodiments for carrying out the present invention are described in detail. The various embodiments are described with reference to the drawings, wherein like reference numerals are used to refer to like elements throughout. In the following description, for purpose of explanation, numerous specific details are set forth in order to provide a thorough understanding of one or more embodiments. It may be evident that such embodiments may be practiced without these specific details. In other instances, well known materials or methods have not been described in detail in order to avoid unnecessarily obscuring embodiments of the present disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the present disclosure. Disclosed embodiments provide method and kit for determining antibiotic susceptibility of micro-organisms

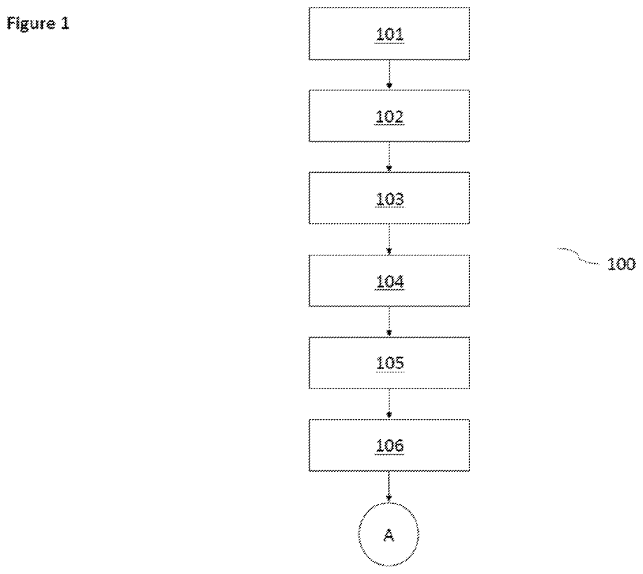

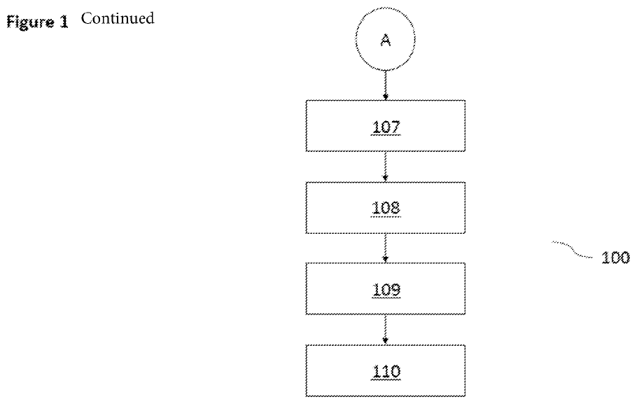

FIG. 1 illustrates a flowchart of a method 100 of determining antibiotic susceptibility associated with a micro-organism, according to a first embodiment. In embodiments, suitable micro-organisms include a method for assessing the drug susceptibility of bacterial cells which may optionally be pathogenic bacteria. The selected organism may be bacteria of a selected genus such as Staphylococcus, Escherichia, Klebsiella, Acinetobacter or Mycobacterium tuberculosis and the selected organism may, for example, be Staphylococcus aureus, Escherichia coli, Klebsiella pneumoniae, Acinetobacter baumannii, and Mycobacterium tuberculosis. In some embodiments the bacteria may be a bacterial isolate or sub-species (e.g from one of the aforementioned species). Optionally, where the cells are bacteria or other microbes or infectious agents the cells may originate (including be derived from) a single individual or host or from multiple individuals or hosts. In embodiments, the bacterial cells may be analyzed or characterized individually or collectively as a colony or group of bacterial cells.

In embodiments, a cell culture including the cells to be tested is obtained or established. The cells may be isolated cells such as isolated cells from a selected organism (e.g. bacteria) which may optionally be from a selected species or genus. Accordingly in one embodiment the method is a method for assessing the drug susceptibility of cells from a selected organism wherein the method includes obtaining or establishing a culture including isolated cells from the selected organism e.g. bacterial cells such as the above-mentioned bacterial species and genera, or as otherwise mentioned herein, or the like.

In embodiments, at step 101, a sample including the micro-organisms is obtained. The sample may be, for example, a biological fluid sample, blood, sputum, urine, cerebrospinal fluid, etc. The sample may be portioned such that a primary portion of the sample and a secondary portion of the sample is created. At step 102, the primary portion of the sample is processed to identify a type of the micro-organism present in the sample. In an embodiment, in identifying the type of the micro-organism, the micro-organisms in the sample may be subjected to lysis. Once lysed, a nucleic acid from the lysed micro-organism is extracted. The nucleic acid may be, for example, deoxyribonucleic acid (DNA) or ribonucleic acid (RNA). In a further embodiment, the extracted nucleic acid may be amplified using polymerase chain reaction (PCR). The polymerase chain reaction may be symmetric or asymmetric. Symmetric PCR may be followed by a TaqMan PCR. For example, the TaqMan PCR may involve use of TaqMan probes (oligonucleotide probe) which improve the specificity of quantitative PCR. TaqMan probes are coupled to or associated with a fluorophore at a 5′ end of the oligonucleotide probe and a quencher at a 3′ end of the oligonucleotide probe. The fluorophore may be, for example, 6-carboxyfluorescein or tetrachlorofluorescein. The quencher may be, for example, tetramethylrhodamine. The quencher molecule quenches a fluorescence emitted by the fluorophore when excited by a light source. During the amplification process, Taq polymerase degrades the probe that has annealed to the nucleic acid associated with the micro-organism. When degraded, the fluorophore is released thereby increasing its distance from the quencher. This causes fluorescence to develop.

In an alternate embodiment, asymmetric PCR may be performed to amplify the nucleic acid associated with the micro-organism. Asymmetric PCR may be followed by PCR melting curve analysis. A temperature at which 50% of the nucleic acid is denatured is identified as a melting temperature (Tₘ). The Tₘ for different micro-organisms is different, thereby enabling effective identification of type of micro-organism present in the sample.

At step 103, the secondary portion of the sample is further divided into at least one first portion and at least one second portion. At step 104, the at least one first portion/s of the sample is incubated with different antibiotics and the at least one second portion of the sample is incubated without any antibiotic. The portion incubated with and without antibiotic/s are equally sized. Therefore, the at least one second portion of the sample may act as a standard against which antibiotic susceptibility of the microorganism is measured. In an embodiment, each of the at least one first portion of the sample may be incubated with a different antibiotic. In an alternate embodiment, a concentration of the antibiotic may vary for different samples. The micro-organisms in the sample are required to be grown/cultured for the shortest possible time in which discernible nucleic acid differences can be measured in the subsequent amplification reaction. For example, the micro-organisms are cultured for a range of at least one to three, one to four, or one to five doubling cycles.

Once the doubling cycle range is met for the at least one first and second portion of the sample, at step 105, the incubated samples are subjected to lysis. From the information obtained from step 102, only those portions incubated with antibiotics relevant for the identified pathogen is taken forward for further processing. The micro-organisms in the sample may be lysed, for example, using enzymatic lysis methods or chemical lysis methods. In an embodiment, lysis of micro-organisms may be performed only for selected at least one first portion of the sample. This may be determined based on the type of micro-organism determined at step 102. Once lysed, at step 106, the nucleic acid associated with the micro-organisms is extracted. The nucleic acid may be DNA or RNA.

At step 107, the nucleic acids are amplified using PCR. In an embodiment, the primers used on the amplification process may be specific to 16srRNA fraction if the extracted nucleic acid is RNA or 16srDNA fraction if the extracted nucleic acid is DNA. Alternatively, the primers may be specific to 18srRNA, 18srDNA, 23srRNA, 23srDNA. Amplification using PCR enables determination of cycle threshold value associated with the amplified nucleic acid. Cycle threshold value is the number of amplification cycles required to reach a level of fluorescence at which a result of PCR changes from ‘not detectable’ to ‘detectable’. At step 108, a first cycle threshold value associated with the amplified nucleic acid from the at least one first portion of the sample is determined. At step 109, a second cycle threshold value associated with the amplified nucleic acid from the at least one second portion of the sample is determined. At step 110, a difference between the first cycle threshold value and the second cycle threshold value is calculated. If the first cycle threshold value is greater than the second cycle threshold value, the antibiotic susceptibility of the micro-organism is lower. In an embodiment, a second PCR melt analysis may be performed to confirm the type of the micro-organism to give species information and confirm that pathogen detected from 102 portion is the one that grew in the presence of antibiotic, thus clearly correlating the antimicrobial susceptibility with pathogen identity. In embodiments, steps 101, 102, 103, 104, 105, 106 are performed in sequential order.

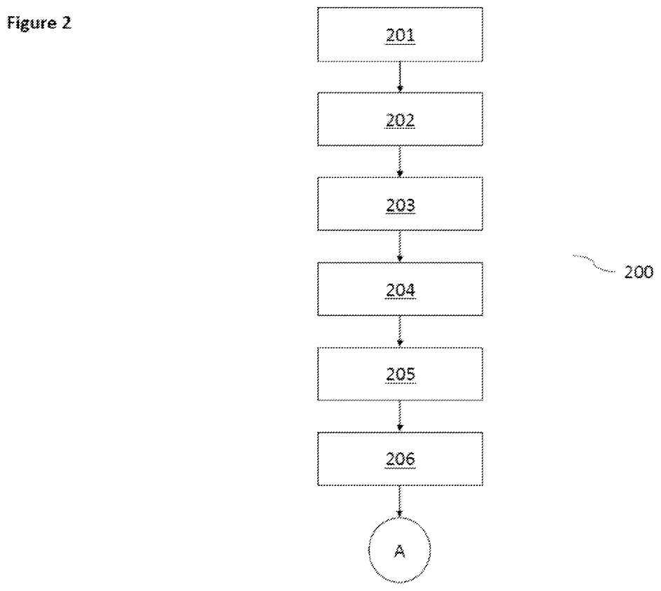

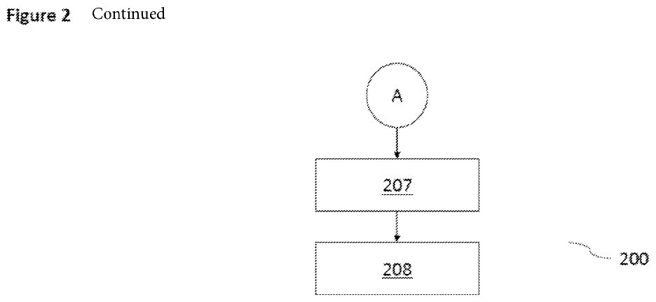

FIG. 2 illustrates a method of determining antibiotic susceptibility of micro-organisms, according to another embodiment. At step 201, the sample including the micro-organism is received. At step 202, at least one first portion of the sample is incubated with an antibiotic and at least one second portion of the sample is incubated with no antibiotic. The micro-organisms in the sample are required to be grown/cultured for the shortest possible time in which discernible nucleic acid differences between both the portions can be measured. For example, the micro-organisms are cultured for a range of at least one to five doubling cycles. For example, the number of doubling cycles may be determined during the development phase and fixed in the actual workflow. Once the doubling cycles are complete for the at least one first and second portion of the sample, at step 203, the incubated samples are subjected to lysis. The micro-organisms in the sample may be lysed, for example, using enzymatic lysis methods or chemical lysis methods. Once lysed, at step 204, the nucleic acid associated with the micro-organisms is extracted and amplified. The nucleic acid may be DNA or RNA. In an embodiment, the primers used on the amplification process may be specific to 16srRNA fraction if the extracted nucleic acid is RNA or 16srDNA fraction if the extracted nucleic acid is DNA. Alternatively, the primers may be specific to 18srRNA, 18srDNA, 23srRNA, 23srDNA.

At step 205, a first cycle threshold value associated with the amplified nucleic acid from the at least one first portion of the sample, in which antibiotic is added, is determined. At step 206, a second cycle threshold value associated with the amplified nucleic acid from the at least one second portion of the sample, in which no antibiotic is added, is determined. At step 207, a difference between the first cycle threshold value and the second cycle threshold value is calculated. If the first cycle threshold value is greater than the second cycle threshold value, the micro-organisms are susceptible to antibiotic in the first portion, and hence the threshold cycle is less than the second portion in which no antibiotic is present. When there is no antibiotic, the micro-organism will double in every doubling cycle and hence the threshold cycle will be lesser, with increasing DNA amount. If the difference between the threshold cycles is negligible or lesser than a set threshold value, the micro-organism incubated with antibiotics in the first portion, is resistant to antibiotics. At step 208, a PCR melt analysis may be performed to confirm the type of the micro-organism. Pathogen identification can be achieved using differently labelled pathogen specific probes of varying Tm. Each probe will bind to a unique pathogen. Pathogen will be identified by a combination of color and Tm. In embodiments, steps 201, 202, 203, 204, 205, 206, 207 and 208 are performed in sequential order.

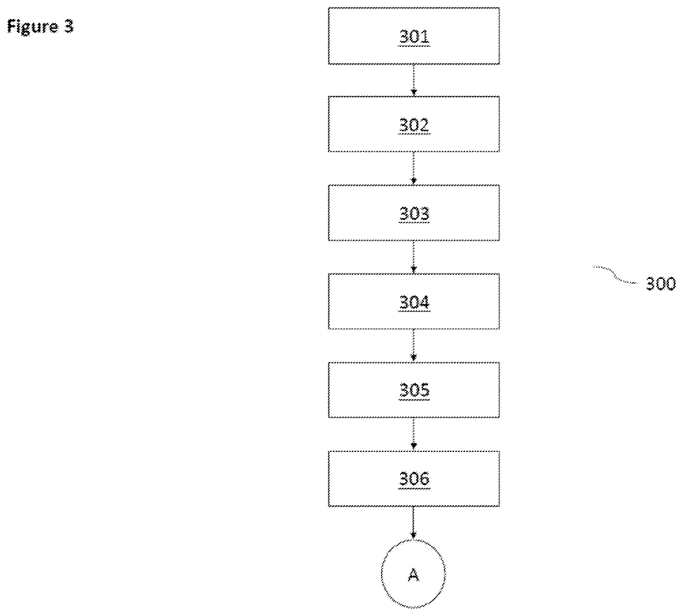

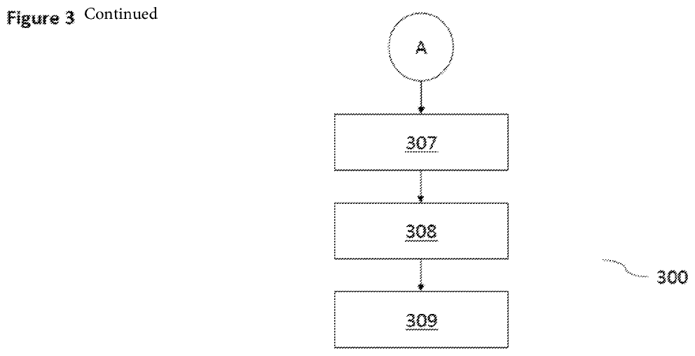

FIG. 3 illustrates a method of determining antibiotic susceptibility of micro-organisms, according to yet another embodiment. At step 301, the sample comprising the micro-organism is received. At step 302, at least one first portion of the sample is incubated with an antibiotic and at least one second portion of the sample is incubated with no antibiotic. The micro-organisms in the sample are required to be grown/cultured or incubated for the shortest possible time in which discernible nucleic acid differences can be measured. For example, the micro-organisms are cultured or incubated for a range of at least one to five doubling cycles. Antibiotics which are cell wall synthesis inhibitors (beta lactam) and cell membrane inhibitors (colistin) affect the integrity of cell wall of the micro-organisms. This facilitates determination of antibiotic susceptibility. Once the doubling cycle range is met for the at least one first and second portion of the sample, at step 303, an intercalating dye is introduced to the at least one first portion of the sample and the at least one second portion of the sample. The samples are incubated for at least five minutes (e.g., about 5 minutes, about 6 minutes, 5-10 minutes) after the intercalating dyes are introduced. For example, the intercalating dye may be ethidium monoazide, ethidium monoazide bromide, propidium monoazide, isomers of these, and the like. Intercalating dyes are known to penetrate dead micro-organisms and cross-link with chromosomal nucleic acid of the dead micro-organisms. Intercalating dyes do not affect live cells.

At step 304, the samples with the intercalating dye are exposed to visible light using a light source for a duration. In embodiments, the light source includes UV light. In embodiments, the light source is applied for a duration sufficient for the intercalation of the dye with the nucleic acid to be made irreversible. The light may be an intense light and the light source may emit light having a wavelength in the range of 465 nm to 475 nm. For example, the light source may be PMA-lite LED Photolysis device. In an embodiment, the samples may be exposed to light for a time duration of 15 to 20 minutes. Advantageously, the light exposure also converts any unbound dye into a compound that can no longer bind to nucleic acids. In an alternate embodiment, the samples may be pelleted to collect micro-organisms and remove the unbound dye.

At step 305, the samples are subjected to lysis. The micro-organisms in the sample may be lysed, for example, using enzymatic lysis methods or chemical lysis methods. Once lysed, at step 306, the nucleic acid associated with the micro-organisms is extracted and amplified. A quantitative PCR is performed. In an embodiment, the primers used on the amplification process may be specific to 16srRNA fraction if the extracted nucleic acid is RNA or 16srDNA fraction if the extracted nucleic acid is DNA. Alternatively, the primers may be specific to 18srRNA, 18srDNA, 23srRNA, 23srDNA.

At step 307, a first cycle threshold value associated with the amplified nucleic acid from the at least one first portion of the sample is determined. At step 308, a second cycle threshold value associated with the amplified nucleic acid from the at least one second portion of the sample is determined. At step 309, a difference between the first cycle threshold value and the second cycle threshold value is calculated. If the first cycle threshold value is greater than the second cycle threshold value, the antibiotic susceptibility of the micro-organism is lower. Micro-organisms that have been exposed to antibiotics will either die (if they are susceptible) or will continue to grow (if they are resistant). Susceptible micro-organisms with compromised cell walls will enable the intercalating dye to penetrate and bind to the chromosomal DNA, thereby rendering it PCR incompatible. Resistant micro-organisms on the other hand will prevent the intercalating dye from penetrating into the cell wall and hence will continue to divide.

Normal human samples contain DNA from dead/dying cells that may skew the results and make it difficult to differentiate between sensitive and resistant micro-organisms in the sample. Advantageously, the intercalating dye also binds with such DNA from dead/dying cells, thereby enabling detection of DNA only from live micro-organisms, by PCR. Further, this enables greater and faster difference between the cycle threshold values associated with the at least one first portion of the sample and the at least one second portion of the sample.

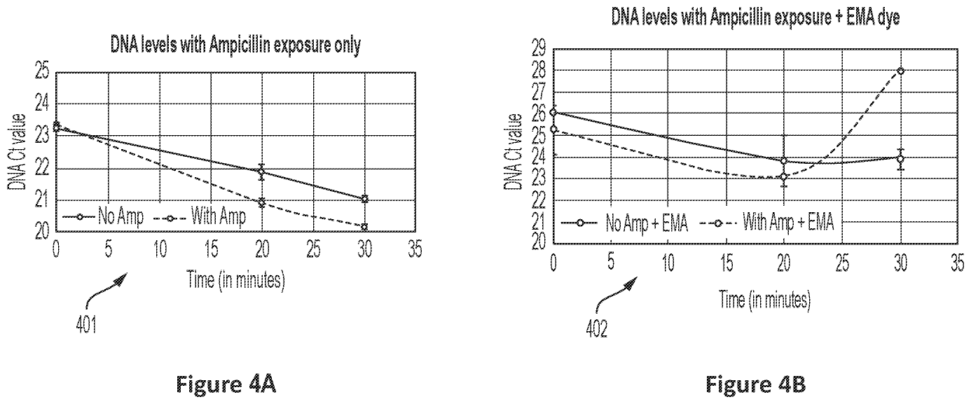

FIG. 4 provides a graphical representation 401, 402 of difference in cycle threshold values of samples which are incubated with antibiotic only and a combination of antibiotic and intercalating dye. In the present embodiment, the antibiotic used is ampicillin (Amp) and the intercalating dye is ethidium monoazide (EMA). Graph 401 illustrates in particular the difference in the cycle threshold values associated with a first portion of the sample incubated with ampicillin and a second portion of the sample incubated with no ampicillin. Graph 402 illustrates in particular the difference in the cycle threshold values associated with a first portion of the sample incubated with ampicillin and EMA and a second portion of the sample incubated with no ampicillin or EMA. It is observed that including EMA in the incubation process improves the difference in the cycle threshold values between the first portion and the second portion of the sample. The EMA seeps into cell membranes of micro-organisms treated with ampicillin and binds to the DNA and makes it PCR incompatible. However, the micro-organisms which are not treated with ampicillin remain intact and the DNA is PCR amplifiable.

In embodiments, the present disclosure includes a method of determining antibiotic susceptibility of a micro-organism in a sample, the method including: receiving the sample containing the micro-organisms; incubating at least one first portion of the sample with at least one antibiotic and at least one second portion of the sample with no antibiotic; contacting the sample such as the at least one first portion and/or at least one second portion with one or more intercalating dyes; extracting nucleic acid from the at least one first portion of the sample and the at least one second portion of the sample, wherein the nucleic acid is associated with the micro-organisms present in the sample; amplifying the extracted nucleic acid from the at least one first portion and the at least one second portion of the sample; and obtaining the antibiotic susceptibility information associated with the micro-organism from the amplified nucleic acid from the at least one first portion and the at least one second portion of the sample. In embodiments the contacting of the intercalating dyes is under conditions sufficient to for the intercalating dye to form a complex with nucleic acid in the sample, or in the microbes.

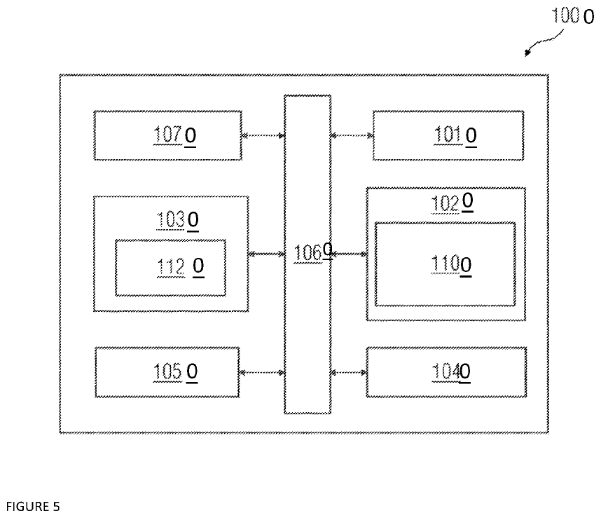

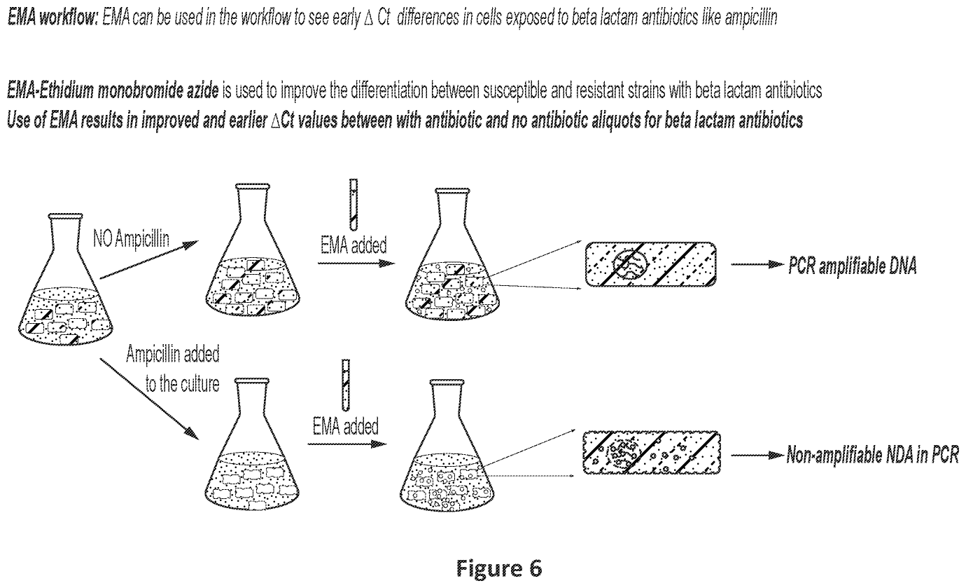

Referring no to FIG. 6, an EMA workflow suitable for use in accordance with the present disclosure is shown. Here, the intercalating dye EMA can be used to see early changes in cycle threshold differences in cells exposed to beta lactam antibiotic like ampicillin. FIG. 6 provides EMA-ethidium monobromide azide used to improve the differentiation between susceptible and resistant strains with beta lactam antibiotics. Use of EMA results in improved and earlier [delta] Ct values (cycle threshold) between with antibiotics and no antibiotic aliquots for beta lactam antibiotics.

Referring now to FIG. 5 a block diagram of system 1000 is shown in which an embodiment can be implemented, for example, as a system 1000 for determining antibiotic susceptibility of a micro-organism, configured to perform the process as described therein. In FIG. 5, the system 1000 includes a processing unit 1010, a memory 1020, a storage unit 1030, an input unit 1040, a bus 1060, an output unit 1050, and a network interface 1070.

The processing unit 1010, as used herein, means any type of computational circuit, such as, but not limited to, a microprocessor, microcontroller, complex instruction set computing microprocessor, reduced instruction set computing microprocessor, very long instruction word microprocessor, explicitly parallel instruction computing microprocessor, graphics processor, digital signal processor, or any other type of processing circuit. The processing unit 1010 may also include embedded controllers, such as generic or programmable logic devices or arrays, application specific integrated circuits, single-chip computers, and the like.

The memory 1020 may be volatile memory and non-volatile memory. The memory 1020 may be coupled for communication with said processing unit 1010. The processing unit 1010 may execute instructions and/or code stored in the memory 1020. A variety of computer-readable storage media may be stored in and accessed from said memory 1020. The memory 1020 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 1020 includes a susceptibility module 1100 stored in the form of machine-readable instructions on any of said above-mentioned storage media and may be in communication to and executed by processor 1010. In embodiments, method steps executed by the processor 1010 to achieve the abovementioned functionality are elaborated upon in detail in FIGS. 1, 2 and/or 3.

The storage unit 1030 may be a non-transitory storage medium which stores a medical database 1120 or other information. The medical database 1120 is a repository of patient data, including blood parameters, that is maintained by a healthcare service provider. The input unit 1040 may include input means such as keypad, touch-sensitive display, camera (such as a camera receiving gesture-based inputs), etc. capable of receiving in-put signal. The bus 106 acts as interconnect between the processing unit 1010, the memory 1020, the storage unit 1030, the input unit 1040, the output unit 1050 and the network interface 1070.

Those of ordinary skilled in the art will appreciate that said hardware depicted in FIG. 5 may vary for particular implementations. For example, other peripheral devices such as an optical disk drive and the like. Local Area Network (LAN)/Wide Area Network (WAN)/Wireless (e.g., Wi-Fi) adapter, graphics adapter, disk controller, input/output (I/O) adapter also may be used in addition or in place of the hardware depicted. Said depicted example is provided for the purpose of explanation only and is not meant to imply architectural limitations with respect to the present disclosure.

The system 1000 in accordance with an embodiment of the present disclosure includes an operating system employing a graphical user interface. Said operating system permits multiple display windows to be presented in the graphical user interface simultaneously with each display window providing an interface to a different application or to a different instance of the same application. A cursor in said graphical user interface may be manipulated by a user through a pointing device. The position of the cursor may be changed and/or an event such as clicking a mouse button, generated to actuate a desired response. One of various commercial operating systems, such as a version of Microsoft Windows™, a product of Microsoft Corporation located in Redmond, Washington may be employed if suitably modified. Said operating system is modified or created in accordance with the present disclosure as described.

Additionally, non-transitory computer readable media containing executable instructions that when executed cause a processor to perform operations including a method as provided herein are provided. In embodiments, the present disclosure includes an article of manufacture, such as a system or component thereof including a non-transitory computer-readable medium with instructions encoded thereon, the instructions configured to cause one or more processors to perform a method including: determining antibiotic susceptibility of a micro-organism in accordance with the present disclosure. In embodiments the present disclosure includes non-transient computer readable medium with instructions encoded thereon, the instructions configured to cause one or more processors to perform a method including: determining antibiotic susceptibility of a micro-organism in accordance with the present disclosure.

In embodiments, the present disclosure includes an article of manufacture, such as a system or component thereof including a non-transitory computer-readable medium with instructions encoded thereon, the instructions configured to cause one or more processors to perform a method comprising: determining antibiotic susceptibility of a micro-organism in a sample, the method comprising: a non-transient computer readable medium with instructions encoded thereon, the instructions configured to cause one or more processors to perform a method (such as methods 100, 200, or 300) of determining antibiotic susceptibility of a micro-organism.

In embodiments, the present disclosure includes an article of manufacture, such as a system or component thereof including a non-transitory computer-readable medium with instructions encoded thereon, the instructions configured to cause one or more processors to perform a method of determining antibiotic susceptibility of a micro-organism in a sample, the method including: incubating at least one first portion of a sample containing micro-organisms with at least one antibiotic and at least one second portion of the sample with no antibiotic; extracting nucleic acid from the at least one first portion of the sample and the at least one second portion of the sample, wherein the nucleic acid is associated with the micro-organisms present in the sample; amplifying the extracted nucleic acid from the at least one first portion and the at least one second portion of the sample; and obtaining the antibiotic susceptibility information associated with the micro-organism from the amplified nucleic acid from the at least one first portion and the at least one second portion of the sample. In embodiments, the method further includes contacting the nucleic acids with one or more intercalating dyes, prior to amplifying or prior to extracting nucleic acid. In embodiments, the method further includes contacting the nucleic acids with one or more intercalating dyes, prior to extracting the nucleic acids.

In embodiments the present disclosure includes a non-transient computer readable medium with instructions encoded thereon, the instructions configured to cause one or more processors to perform a method of determining antibiotic susceptibility of a micro-organism in a sample, the method including: incubating at least one first portion of a sample containing micro-organisms with at least one antibiotic and at least one second portion of the sample with no antibiotic; extracting nucleic acid from the at least one first portion of the sample and the at least one second portion of the sample, wherein the nucleic acid is associated with the micro-organisms present in the sample; contacting the nucleic acid with one or more intercalating dyes under conditions to form a nucleic acid/intercalating dye complex; amplifying the extracted nucleic acid from the at least one first portion and the at least one second portion of the sample; and obtaining the antibiotic susceptibility information associated with the micro-organism from the amplified nucleic acid from the at least one first portion and the at least one second portion of the sample. In embodiments, contacting the nucleic acid with one or more intercalating dyes under conditions to form a nucleic acid/intercalating dye complex occurs prior to extraction. In embodiments, contacting the nucleic acid with one or more intercalating dyes under conditions to form a nucleic acid/intercalating dye complex occurs prior to amplification.

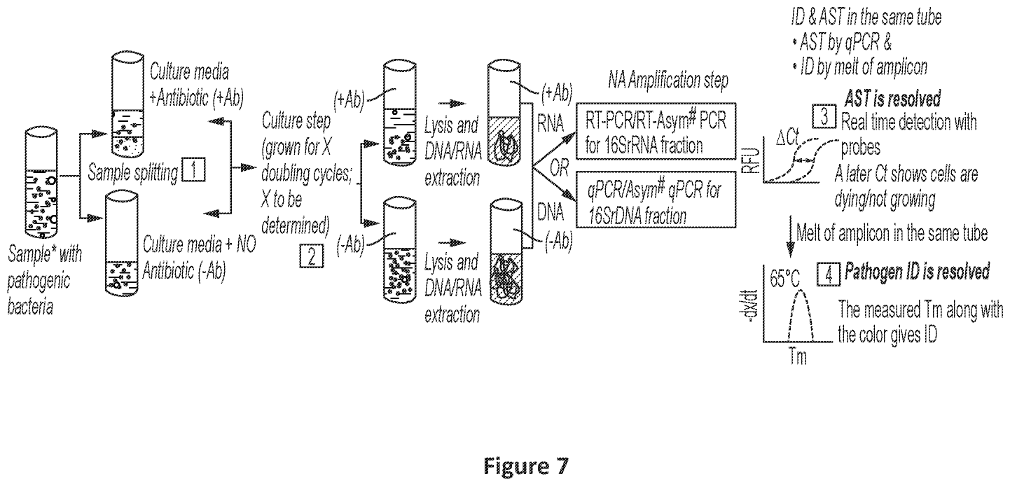

Referring now to FIG. 7, a method of determining antibiotic susceptibility of micro-organisms, according to yet another embodiment is shown. Here, a culturing step is shown, followed by extracting, and amplifying in accordance with the present disclosure. AST is shown as resolved, and pathogen ID is resolved using Tm information and color coded identification.

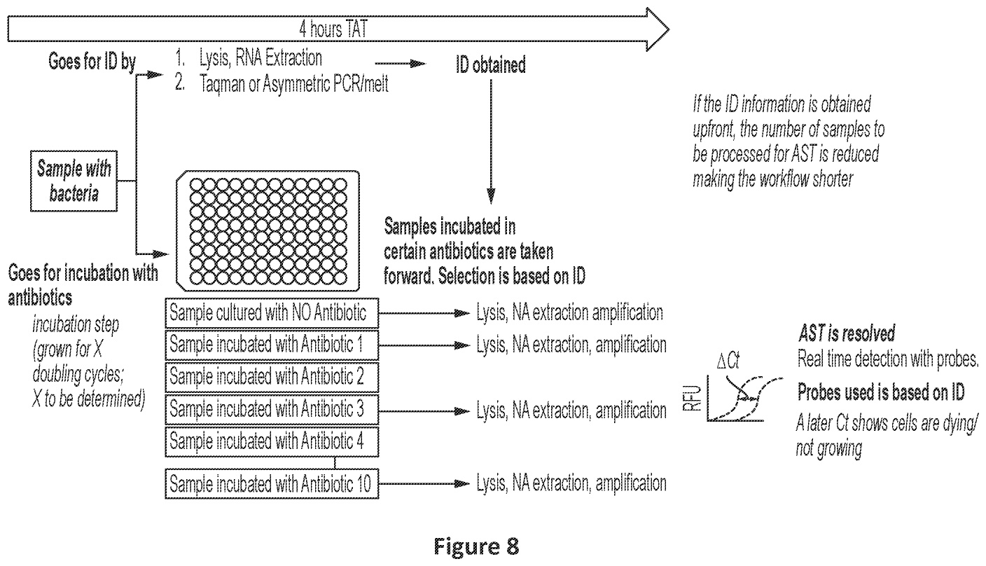

Referring now to FIG. 8, a method of determining antibiotic susceptibility of micro-organisms, according to yet another embodiment is shown. Here the duration of the workflow is reduced by obtaining bacterial identification upfront in accordance with FIG. 8.

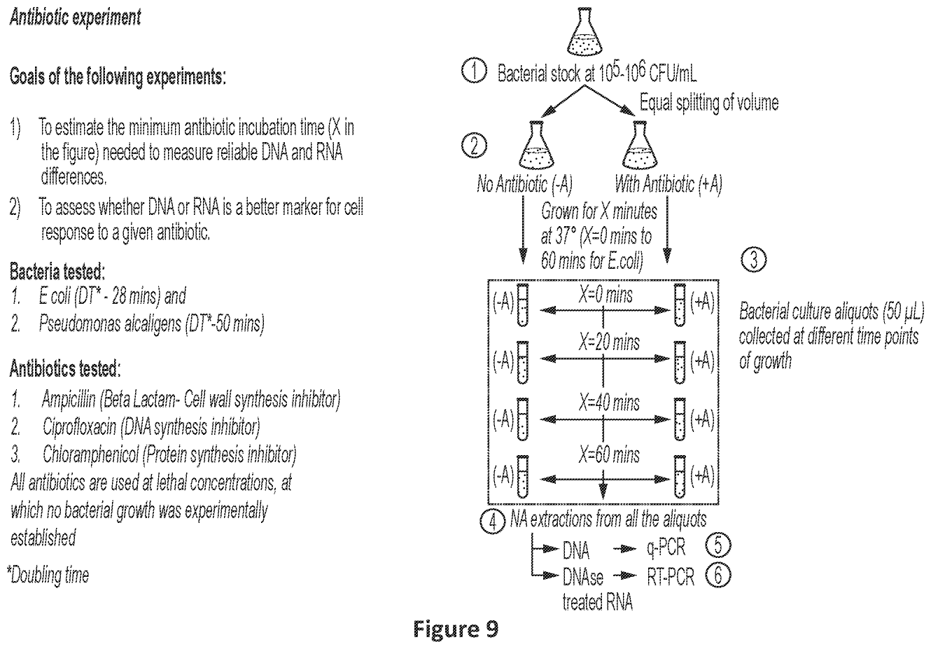

FIG. 9 depicts a method of determining antibiotic susceptibility of micro-organisms, according to yet another embodiment.

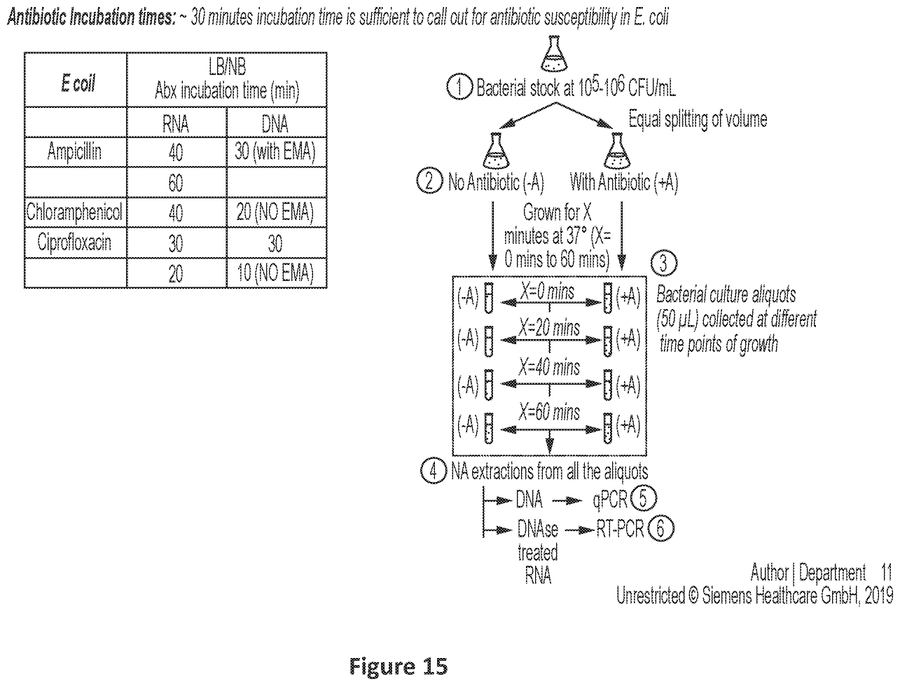

FIG. 15 depicts antibiotic incubation times in accordance with embodiments of the present disclosure. FIG. 15 demonstrates that about 30 minutes incubation time is sufficient to call out for antibiotic susceptibility in E.coli.

### Treatment

In embodiments, the present disclosure includes a method of treating a subject in need thereof by determining that the subject is a subject in need thereof (such as has a pathogenic bacterial infection, or sepsis), and subsequently treating the subject. In embodiments, the methods, systems and kits described herein are suitable for use by a health care provider in a point of care setting. For examples, the methods of the present disclosure can be applied as a diagnostic test to diagnose a patient or subject as positive for a pathogenic bacterial infection or sepsis, followed by administering a therapeutic agent or compound to the subject in need thereof in a therapeutically effective amount. For example, a physician may administer a therapeutically effective amount of any drug, therapeutic agent or biologic suitable for treating a bacterial infection, or disease state subsequent to diagnosis in accordance with the present disclosure. One non-limiting example of an agent suitable for treating a bacterial infection includes one or more antibiotics in accordance with the present disclosure. The therapeutically effective amount of agent provided may be determined by a physician based on the subject's response, comorbidities, and the like. In embodiments, a therapeutic compound is administered in an effective amount to a subject in a diseased state. In embodiments, a therapeutically effective amount of a compound such as antibiotic is administered to a subject in need thereof in an amount sufficient to change the natural or presenting state of the subject.

In certain embodiments, the disease treated in accordance with the methods described herein is a disease caused by bacterial infection. Non-limiting examples of disease-causing bacteria include: Streptococcus pneumoniae, Mycobacterium tuberculosis, Chlamydia pneumoniae, Bordetella pertussis, Mycoplasma pneumoniae, Haemophilus influenzae, Moraxella catarrhalis, Legionella, Pneumocystis jiroveci, Chlamydia psittaci, Chlamydia trachomatis, Bacillus anthracis, and Francisella tularensis, Borrelia burgdorferi, Salmonella, Yersinia pestis, Shigella, E. coli, Corynebacterium diphtheriae, and Treponema pallidum.

In certain embodiments, a composition such as an antibiotic is administered to a patient who has been diagnosed with a disease caused by infection with a bacteria, e.g., the patient has been infected by Streptococcus pneumoniae, Mycobacterium tuberculosis, Chlamydia pneumoniae, Bordetella pertussis, Mycoplasma pneumoniae, Haemophilus influenzae, Moraxella catarrhalis, Legionella, Pneumocystis jiroveci, Chlamydia psittaci, Chlamydia trachomatis, Bacillus anthracis, and Francisella tularensis, Borrelia burgdorferi, Salmonella, Yersinia pestis, Shigella, E. coli, Corynebacterium diphtheriae, and/or Treponema pallidum.

In certain embodiments, the disease treated in accordance with the methods described herein is a disease caused by bacterial infection. Non-limiting examples of disease-causing bacteria include: Streptococcus pneumoniae, Mycobacterium tuberculosis, Chlamydia pneumoniae, Bordetella pertussis, Mycoplasma pneumoniae, Haemophilus influenzae, Moraxella catarrhalis, Legionella, Pneumocystis jiroveci, Chlamydia psittaci, Chlamydia trachomatis, Bacillus anthracis, and Francisella tularensis, Borrelia burgdorferi, Salmonella, Yersinia pestis, Shigella, E. coli, Corynebacterium diphtheriae, and Treponema pallidum.

In certain embodiments, a composition is administered to a patient who has been diagnosed with a disease caused by infection with a bacteria, e.g., the patient has been infected by Streptococcus pneumoniae, Mycobacterium tuberculosis, Chlamydia pneumoniae, Bordetella pertussis, Mycoplasma pneumoniae, Haemophilus influenzae, Moraxella catarrhalis, Legionella, Pneumocystis jiroveci, Chlamydia psittaci, Chlamydia trachomatis, Bacillus anthracis, and Francisella tularensis, Borrelia burgdorferi, Salmonella, Yersinia pestis, Shigella, E. coli, Corynebacterium diphtheriae, and/or Treponema pallidum.

### EXEMPLARY NUMBERED EMBODIMENTS

Embodiment 1. A method of determining antibiotic susceptibility of a micro-organism in a sample, the method comprising: receiving the sample containing the micro-organisms; incubating at least one first portion of the sample with at least one antibiotic and at least one second portion of the sample with no antibiotic; extracting nucleic acid from the at least one first portion of the sample and the at least one second portion of the sample, wherein the nucleic acid is associated with the micro-organisms present in the sample; amplifying the extracted nucleic acid from the at least one first portion and the at least one second portion of the sample; and obtaining the antibiotic susceptibility information associated with the micro-organism from the amplified nucleic acid from the at least one first portion and the at least one second portion of the sample.

Embodiment 2. A method of embodiment 1, further including identifying a type of the micro-organism present in the sample.

Embodiment 3. A method of embodiments 1 or 2, further comprising: lysing the micro-organisms present in the sample; extracting nucleic acids associated with the micro-organisms; amplifying the extracted nucleic acids using polymerase chain reaction; and identifying the type of micro-organism present in the sample based on the amplified nucleic acids, wherein the amplified nucleic acids are subjected to a polymerase chain reaction based melting analysis.

Embodiment 4. A method of any embodiments 1-3, further comprising: lysing the micro-organisms present in the sample; extracting nucleic acids associated with the micro-organisms; amplifying the extracted nucleic acids using polymerase chain reaction; and identifying the type of micro-organism present in the sample based on the amplified nucleic acids, wherein the nucleic acids are amplified using a probe coupled to a fluorophore, wherein the probe is complementary to a target nucleic acid sequence associated with the micro-organism, and wherein the type of micro-organism is identified based on a color emitted by the fluorophore and the Tm of the target with bound probe. The probes further can have varying Tm values.

Embodiment 5. A method of any embodiments 1-4, further comprising: incubating the at least one first portion of the sample and the at least one second portion of the sample with an intercalating dye; and exposing the incubated samples to visible light using a light source.

Embodiment 6. A method of any embodiments 1-5, wherein the intercalating dyes is at least one of ethidium monoazide, ethidium monoazide bromide, and propidium monoazide.

Embodiment 7. A method of any embodiments 1-6, wherein obtaining the antibiotic susceptibility information associated with the micro-organism from the amplified nucleic acid comprises: determining a first cycle threshold value associated with the amplified nucleic acid from the at least one first portion; determining a second cycle threshold value associated with the amplified nucleic acid from the at least one second portion of the sample; calculating a difference between the first cycle threshold value and the second cycle threshold value; and determining the antibiotic susceptibility information based on the cycle threshold value associated with the amplified nucleic acid based on the difference between the first cycle threshold value and the second cycle threshold value, wherein if the cycle threshold value is higher, the antibiotic susceptibility of the micro-organism is lower.

Embodiment 9. A method of any embodiments 1-8, further comprising determining the identity of the micro-organism after obtaining the antibiotic susceptibility information associated with the micro-organism.

Embodiment 10. A method of any embodiments 1-9, wherein the at least one first portion of the sample is incubated with more than one type of antibiotics.

Embodiment 11. A method of any embodiments 1-9, wherein the at least one first portion of the sample is incubated with more than one type of antibiotics, wherein the antibiotics are present in varying concentrations.

Embodiment 12. A kit for determining antibiotic susceptibility of a micro-organism in a sample including components of embodiments 1-11.

Embodiment 13. A kit for determining antibiotic susceptibility of a micro-organism in a sample, the kit comprising: one or more antibiotics; growth media for micro-organisms; reagents associated with nucleic acid extraction; reagents associated with nucleic acid amplification; and polymerase chain reaction based melt analysis software.

Embodiment 14. A kit of embodiments 12 or 13, further comprising one or more intercalating dyes.

Embodiment 15. A method of treating a subject in need thereof including determining antibiotic susceptibility of a micro-organism in accordance with an embodiment of the present disclosure and subsequently treating the subject in need thereof. In embodiments, treating includes administering to the subject a therapeutically effective amount of a composition such as one or more antibiotics.

Embodiment 16. A non-transient computer readable medium with instructions encoded thereon, the instructions configured to cause one or more processors to perform a method of determining antibiotic susceptibility of a micro-organism in a sample, the method including: incubating at least one first portion of a sample containing micro-organisms with at least one antibiotic and at least one second portion of the sample with no antibiotic; extracting nucleic acid from the at least one first portion of the sample and the at least one second portion of the sample, wherein the nucleic acid is associated with the micro-organisms present in the sample; contacting the nucleic acid with one or more intercalating dyes under conditions to form a nucleic acid/intercalating dye complex; amplifying the extracted nucleic acid from the at least one first portion and the at least one second portion of the sample; and obtaining the antibiotic susceptibility information associated with the micro-organism from the amplified nucleic acid from the at least one first portion and the at least one second portion of the sample.

Embodiment 17. A method of treating a subject in need thereof including determining antibiotic susceptibility of a micro-organism in a biological sample from a subject, the method comprising: receiving the sample containing the micro-organisms; incubating at least one first portion of the sample with at least one antibiotic and at least one second portion of the sample with no antibiotic; extracting nucleic acid from the at least one first portion of the sample and the at least one second portion of the sample, wherein the nucleic acid is associated with the micro-organisms present in the sample; amplifying the extracted nucleic acid from the at least one first portion and the at least one second portion of the sample; and obtaining the antibiotic susceptibility information associated with the micro-organism from the amplified nucleic acid from the at least one first portion and the at least one second portion of the sample, and subsequently treating the subject in need thereof. In embodiments, treating includes administering to the subject a therapeutically effective amount of a composition. In embodiments, the disease is characterized as bacterial disease, or the like.

The terms “treatment” or “treating” of a subject includes the application or administration of a compound to a subject with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being. In embodiments, the term “treating” means reducing or ameliorating the progression, severity, and/or duration of bacterial infection, or ameliorating one or more symptoms of bacterial infection caused by administration of one or more therapies (e.g., one or more therapeutic agents). In a particular embodiment, the term “treatment” means to ameliorate measurable physical parameters of bacterial infection. In embodiments, the term “treating” means reducing or ameliorating the progression, severity, and/or duration of a bacterial infection or ameliorating one or more symptoms of a bacterial caused by administration of one or more therapies (e.g., one or more therapeutic agents). In a particular embodiment, the term “treatment” means to ameliorate measurable physical parameters of a bacterial infection. In embodiments, the term “treating” means reducing or ameliorating the progression, severity, and/or duration of sepsis, or ameliorating one or more symptoms of a sepsis caused by administration of one or more therapies (e.g., one or more therapeutic agents). In a particular embodiment, the term “treatment” means to ameliorate measurable physical parameters of sepsis. In embodiments, “treating” changes the natural or presenting state of a subject.

As used herein, the term “therapeutically effective amount” means the amount of compound that, when administered to a subject for treating or preventing a particular disorder, disease or condition, is sufficient to effect such treatment or prevention of that disorder, disease or condition. Dosages and therapeutically effective amounts may vary for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g., bioavailability, stability, potency, toxicity, etc.), and the particular disorder(s) the subject is suffering from. In addition, the therapeutically effective amount that is administered intravenously may depend on the subject's blood parameters e.g., lipid profile, insulin levels, glycemia or liver metabolism. The therapeutically effective amount will also vary according to the severity of the disease state, organ function, or underlying disease or complications. Such appropriate doses may be determined using any available assays. When one or more of the compounds or therapeutic agents is to be administered to humans, a physician may for example, prescribe a relatively low dose at first, subsequently increasing the dose until an appropriate response is obtained.

### EXAMPLES

FIG. 9 shows an experimental process flow outlining the goals of experiments described hereinbelow, bacteria test and antibiotics tested in accordance with the present disclosure.

### Example 1: Detection on Bacterial Sensitivity to Ampicillin

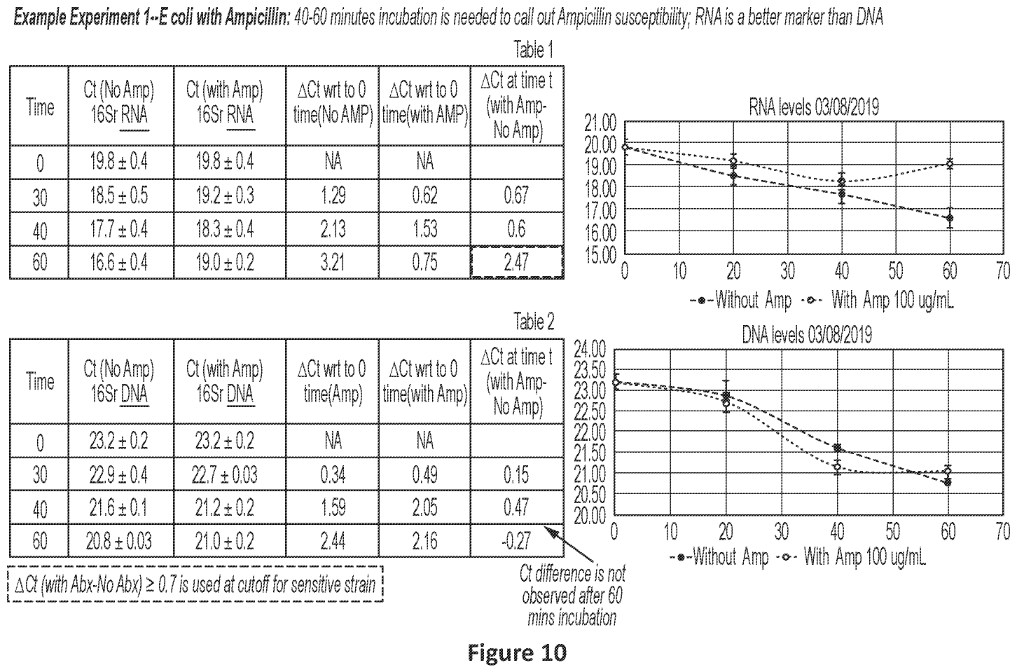

Overnight E. coli culture that was susceptible to ampicillin was further sub-cultured for 2-3 hours to log phase. Cells in this culture were quantified using OD₆₀₀ₙₘ values. The culture was diluted to 1×10⁶ cells/mL and 10 mL was aliquoted into two, 50 ml conical flask. Ampicillin at 100 μg/mL was added to one of the conical flasks. The two flasks, with and without antibiotic were incubated at 37° C. and two, 50 μL aliquots were taken from each flask at regular intervals (0, 30, 40 and 60 mins). Nucleic acid (NA) was extracted from all sample aliquots using the VERSANT® brand Sample preparation 1.0 reagent kit. The NA samples were then subjected to PCR and RT-PCR (reverse transcription-PCR) using 16SrDNA specific primers (each DNA extracted sample was split into two replicates for PCR). As seen the FIG. 10, table 1, RNA levels was able to detect for ampicillin susceptibility at 60 minutes, whereas DNA levels did not distinguish antibiotic and no antibiotic samples even with 60 min incubation (FIG. 10 table 2). In this experiment, all RNA samples were treated with DNAse for 60 min at 37° C. before RT-PCR. In a subsequent experiment it was shown that a treatment as low as 5 minutes was also sufficient to eliminate DNA from the sample.

### Example 2: Use of EMA to Speed Up Detection on Bacterial Sensitivity to Ampicillin

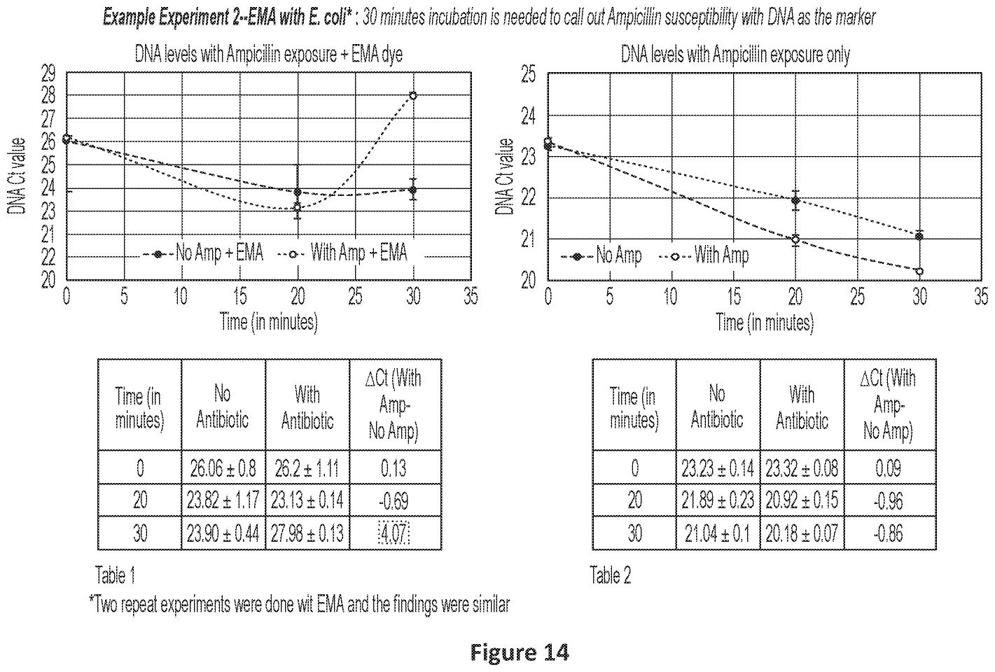

Overnight E. coli culture that was susceptible to ampicillin was further sub-cultured for 2-3 hours to log phase. Cells in this culture was quantified using OD₆₀₀ₙₘ values. The culture was diluted to 1×10⁶ cells/mL and 10 mL was aliquoted into two, 50 ml conical flasks. Ampicillin at 100 μg/mL was added to one of the conical flasks. The two flasks, with and without antibiotic were incubated at 37° C. and four 50 μL aliquots were taken from each flask at regular intervals (0, 20 and 30 mins). 25 μL of 2 μM of EMA (ethidium monoazide made in 20% DMSO) were added into 2 of 4 aliquots from each sampling. After EMA addition these sample were mixed by vortexing, incubated in dark for 5 minutes and transferred into a 1.5 ml Eppendorf tube and exposed to blue LED light for 15 minutes for the photolysis reaction to take place. DNA was extracted from all sample aliquots using the Versant® Sample preparation reagents. The DNA samples were then subjected to PCR using 16SrDNA specific primers (each DNA extracted sample was split into two replicates). As seen in FIG. 14, table 1, samples that were exposed to EMA had a large difference in Ct between ampicillin exposed and unexposed within 30 minutes. The same samples when not exposed to EMA+blue light did not show any difference between the ampicillin exposed and unexposed samples (FIG. 14, table 2).

### Additional Examples

Experiments similar to those described above were performed.

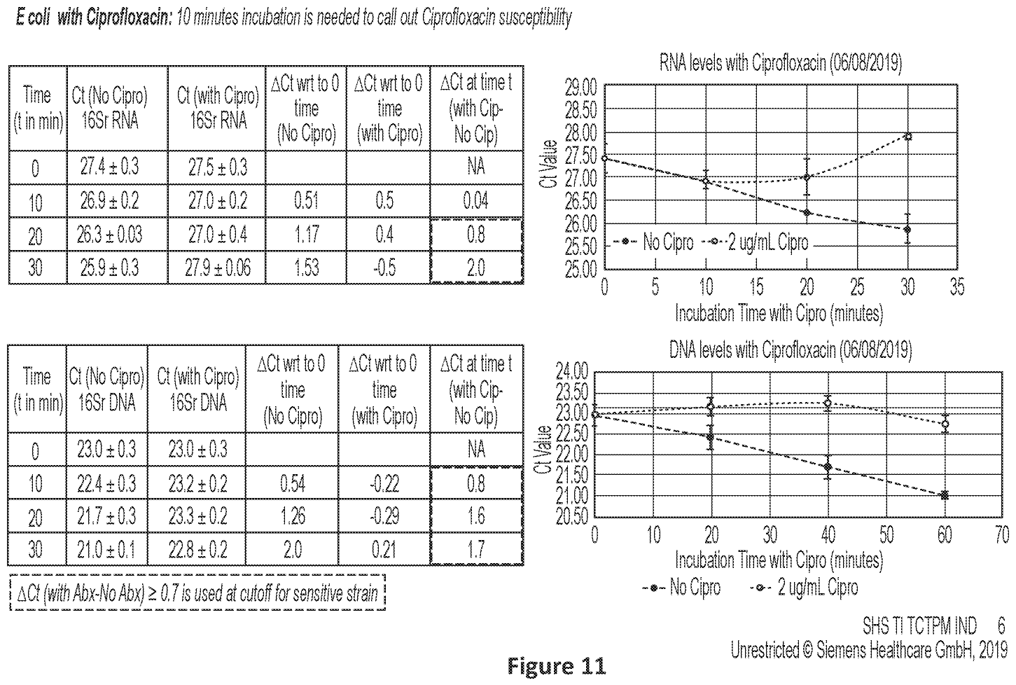

FIG. 11 depicts data relating to E. coli and ciprofloxacin obtained according to an embodiment of the present disclosure.

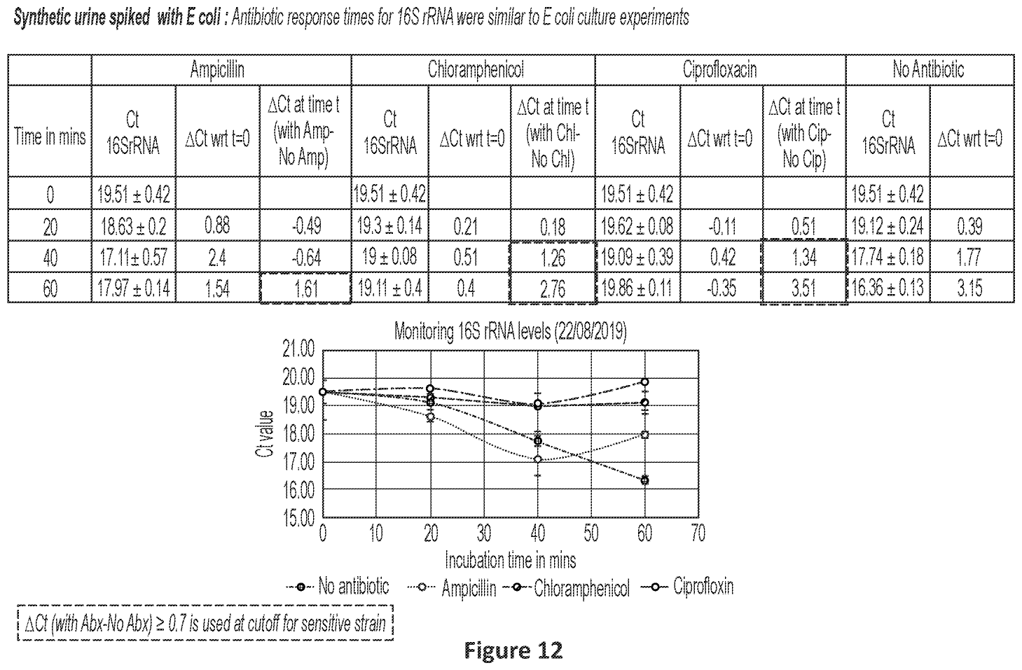

FIG. 12 depicts data relating to E. coli and ciprofloxacin obtained according to an embodiment of the present disclosure.

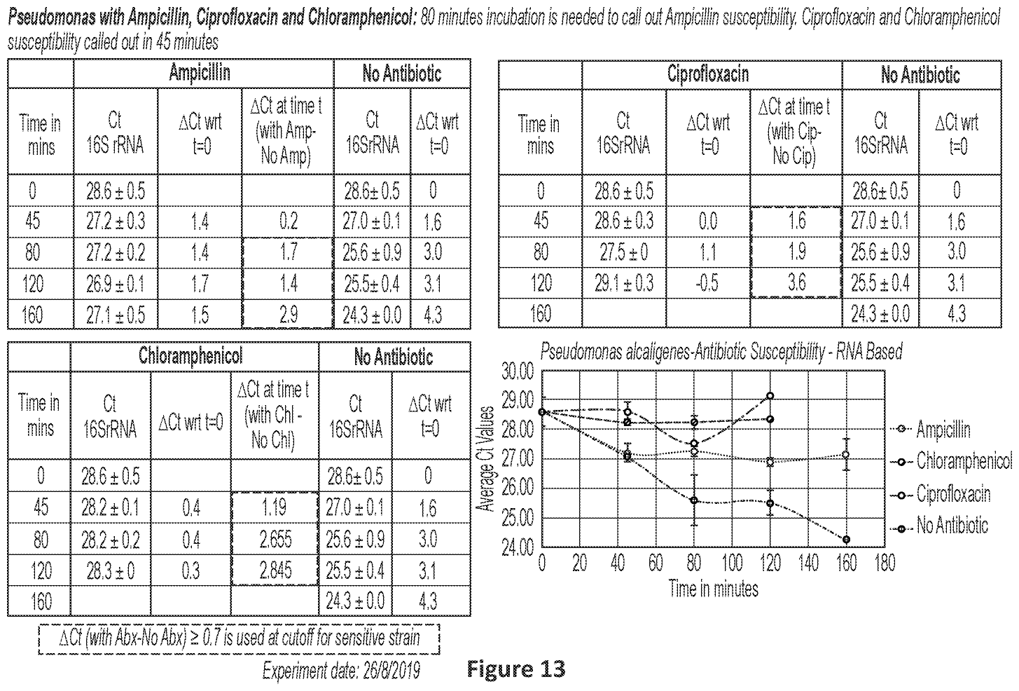

FIG. 13 depicts data relating to pseudomonas with ciprofloxacin and chloramphenicol obtained according to an embodiment of the present disclosure.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials, and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may effect numerous modifications thereto and changes may be made without departing from the scope and spirit of the invention in its aspects.

1. A method of determining antibiotic susceptibility of a micro-organism in a sample, the method comprising:
receiving the sample containing the micro-organisms;
incubating at least one first portion of the sample with at least one antibiotic and at least one second portion of the sample with no antibiotic;
extracting nucleic acid from the at least one first portion of the sample and the at least one second portion of the sample, wherein the nucleic acid is associated with the micro-organisms present in the sample;
amplifying the extracted nucleic acid from the at least one first portion and the at least one second portion of the sample; and
obtaining the antibiotic susceptibility information associated with the micro-organism from the amplified nucleic acid from the at least one first portion and the at least one second portion of the sample.

2. The method according to claim 1, further comprising identifying a type of the micro-organism present in the sample.

3. The method according to claim 2, further comprising:
lysing the micro-organisms present in the sample;
extracting nucleic acids associated with the micro-organisms;
amplifying the extracted nucleic acids using polymerase chain reaction; and
identifying the type of micro-organism present in the sample based on the amplified nucleic acids, wherein the amplified nucleic acids are subjected to a polymerase chain reaction-based melting analysis.

4. The method according to claim 2, further comprising:
lysing the micro-organisms present in the sample;
extracting nucleic acids associated with the micro-organisms;
amplifying the extracted nucleic acids using polymerase chain reaction; and
identifying the type of micro-organism present in the sample based on the amplified nucleic acids, wherein the nucleic acids are amplified using one or more probes coupled to a fluorophore, wherein the one or more probes are complementary to a target nucleic acid sequence associated with the micro-organism, and wherein the type of micro-organism is identified based on a color emitted by the fluorophore and the Tm of the target with bound probe.

5. The method of claim 4, wherein the one or more probes have varying Tm values.

6. The method according to claim 1, further comprising:
incubating the at least one first portion of the sample and the at least one second portion of the sample with an intercalating dye; and
exposing the incubated samples to visible light using a light source.

7. The method according to claim 6, wherein the intercalating dyes is at least one of ethidium monoazide, ethidium monoazide bromide, propidium monoazide, and combinations thereof.

8. The method according to claim 1, wherein obtaining the antibiotic susceptibility information associated with the micro-organism from the amplified nucleic acid comprises:
determining a first cycle threshold value associated with the amplified nucleic acid from the at least one first portion;
determining a second cycle threshold value associated with the amplified nucleic acid from the at least one second portion of the sample;
calculating a difference between the first cycle threshold value and the second cycle threshold value; and
determining the antibiotic susceptibility information based on the cycle threshold value associated with the amplified nucleic acid based on the difference between the first cycle threshold value and the second cycle threshold value, wherein if the cycle threshold value is higher, the antibiotic susceptibility of the micro-organism is lower.

9. The method according to claim 1, further comprising determining the identity of the micro-organism after obtaining the antibiotic susceptibility information associated with the micro-organism.

10. The method according to claim 1, wherein the at least one first portion of the sample is incubated with more than one type of antibiotics.

11. The method according to claim 1, wherein the at least one first portion of the sample is incubated with more than one type of antibiotics, wherein the antibiotics are present in varying concentrations.

12. A kit for determining antibiotic susceptibility of a micro-organism in a sample, the kit comprising:
one or more antibiotics;
growth media for micro-organisms;
reagents associated with nucleic acid extraction;
reagents associated with nucleic acid amplification; and
polymerase chain reaction based melt analysis software.

13. The kit according to claim 12, further comprising one or more intercalating dyes.

14. An article of manufacture including a non-transitory computer-readable medium with instructions encoded thereon, the instructions configured to cause one or more processors to perform a method of determining antibiotic susceptibility of a micro-organism in a sample, the method comprising:
incubating at least one first portion of a sample containing micro-organisms with at least one antibiotic and at least one second portion of the sample with no antibiotic;
extracting nucleic acid from the at least one first portion of the sample and the at least one second portion of the sample, wherein the nucleic acid is associated with the micro-organisms present in the sample;
amplifying the extracted nucleic acid from the at least one first portion and the at least one second portion of the sample; and
obtaining the antibiotic susceptibility information associated with the micro-organism from the amplified nucleic acid from the at least one first portion and the at least one second portion of the sample.

15. A non-transient computer readable medium with instructions encoded thereon, the instructions configured to cause one or more processors to perform a method of determining antibiotic susceptibility of a micro-organism in a sample, the method comprising:
incubating at least one first portion of a sample containing micro-organisms with at least one antibiotic and at least one second portion of the sample with no antibiotic;
extracting nucleic acid from the at least one first portion of the sample and the at least one second portion of the sample, wherein the nucleic acid is associated with the micro-organisms present in the sample;
contacting the nucleic acid with one or more intercalating dyes under conditions to form a nucleic acid/intercalating dye complex;
amplifying the extracted nucleic acid from the at least one first portion and the at least one second portion of the sample; and
obtaining the antibiotic susceptibility information associated with the micro-organism from the amplified nucleic acid from the at least one first portion and the at least one second portion of the sample.

16. A non-transient computer readable medium with instructions encoded thereon, the instructions configured to cause one or more processors to perform a method of determining antibiotic susceptibility of a micro-organism in a sample, the method comprising:
incubating at least one first portion of a sample containing micro-organisms with at least one antibiotic and at least one second portion of the sample with no antibiotic;
contacting the sample or portions thereof with one or more intercalating dyes;
extracting nucleic acid from the at least one first portion of the sample and the at least one second portion of the sample, wherein the nucleic acid is associated with the micro-organisms present in the sample;
amplifying the extracted nucleic acid from the at least one first portion and the at least one second portion of the sample; and
obtaining the antibiotic susceptibility information associated with the micro-organism from the amplified nucleic acid from the at least one first portion and the at least one second portion of the sample.
